(19) United States
(12) Patent Application Publication
(10) Pub. No.: US 2012/0020955 A1
(43) Pub. Date: Jan. 26, 2012

(54) Detection of Neurological Disorders With Immune Reactions

(76) Inventors: Mehdi Tafti, Chene-Bougeries (CH); Michel Muhlethaler, Geneva (CH)

(21) Appl. No.: 13/168,428

(22) Filed: Jun. 24, 2011

Related U.S. Application Data
(60) Provisional application No. 61/358,655, filed on Jun. 25, 2010.

(51) Int. Cl. A61K 39/395; A61K 38/21; C40B 30/04; A61P 37/06; C12Q 1/68; G01N 33/53; A61K 38/20

(52) U.S. Cl. 424/131.1; 436/501; 424/85.6; 424/85.2; 435/7.92; 435/6.12; 506/9

(57) ABSTRACT

The present invention relates generally to the field of methods of diagnosing a neurological disorder with immune reaction in a mammal. Also disclosed are methods of treating and/or preventing a neurological disorder with immune reaction in a patient.

### CROSS-REFERENCE TO RELATED APPLICATIONS

This application claims priority to and the benefit of co-pending U.S. provisional patent application Ser. No. 61/358,655 entitled “DETECTION OF NEUROLOGICAL DISORDERS WITH IMMUNE REACTIONS,” filed Jun. 25, 2010, which is incorporated herein by reference in its entirety.

### FIELD OF THE INVENTION

The present invention relates generally to the field of methods of diagnosing a neurological disorder with immune reaction in a mammal. Also disclosed are methods of treating and/or preventing a neurological disorder with immune reaction in a patient.

### BACKGROUND OF THE INVENTION

Hypocretin (orexin) neurons play a critical role in the regulation of sleep and wakefulness, and disturbances of the hypocretin system have been directly linked to narcolepsy in animals and humans (1-6). Human narcolepsy is believed to be caused by a selective hypocretin neuronal loss (2, 3). Current hypotheses suggest an autoimmune process targeting these neurons. Attempts to characterize immune-related processes have failed so far.

Narcolepsy is tightly associated with the HLA system with 95% of narcolepsy patients with cataplexy carrying the HLA-DQB1*0602 allele and having undetectable hypocretin levels in their cerebrospinal fluid (CSF) (7, 8). A recent genome-wide association study found also a strong association between narcolepsy and a T-cell receptor alpha gene variant, corroborating the autoimmune hypothesis (9). Moreover, using a model of spontaneous colonic migrating motor complex, the presence of functional autoantibodies in sera of narcolepsy patients could be demonstrated (10, 11).

However, peripheral or central immune abnormalities in narcolepsy, even in patients diagnosed shortly after the disorder onset, could not be demonstrated thus far (12). One hypothesis is that hypocretin neurons express a specific peptide recognized as an autoantigen. The autoimmune attack may be acute and narcolepsy symptoms may develop once hypocretin neurons are lost with autoantibody titers below detectable levels. Two peptides neuronal pentraxin 2 (NPTX2 or Narp) and PDYN (Dynorphin), which are co-localized with hypocretin within the posterior lateral hypothalamus, were like hypocretin absent in the few post-mortem brains of narcolepsy patients (13). However, because a putative immune attack does not seem to target either hypocretin ligands or their receptors (12) and because Narp and Dynorphin are abundantly expressed in many brain structures, they are unlikely autoantigen targets.

Therefore is a need to develop improved method of diagnosing neurological disorder with immune reaction.

### SUMMARY OF THE INVENTION

The present invention provides a method of diagnosing a neurological disorder with immune reactions in a mammal comprising the step of detecting, in a test sample from the mammal, autoantibodies to an epitope of at least one Trib2 protein or an antigenic determinant thereof, optionally combined with a quantitative measure of orexins. Also provided is a method of treating and/or preventing a neurological disorder with immune reaction in a patient. Also provided is an assay kit for diagnosing neurological sleep disorders in a mammal.

In one embodiment, a method is provided of diagnosing a neurological disorder with immune reaction in a mammal. The method can comprise the step of detecting, in a test sample from a mammal, autoantibodies to an epitope of at least one Trib2 protein or an antigenic determinant thereof.

In another embodiment, the method of diagnosing a neurological disorder with immune reaction comprises the steps of a) contacting the test sample with the at least one Trib2 protein or an antigenic determinant thereof; and b) detecting any antibody-antigen complexes formed between the at least one Trib2 protein or an antigenic determinant thereof and antibodies present in the test sample, wherein presence of said complex is indicative of a neurological disorder with immune reaction.

In another embodiment, the neurological disorder with immune reaction is selected from the group consisting of narcolepsy with and without cataplexy, idiopathic hypersomnia, Kleine Levin syndrome, multiple sclerosis, Parkinson's disease, Alzheimer's disease, Guillain-Barré syndrome, uveitis, Myasthenia gravis, Neuromyotonia, Neuropathic diabetes, Schizophrenia, and other neuroinflammatory disorders.

In another embodiment, the method comprises the step of detecting orexin A protein and/or orexin B protein in a test sample from said mammal.

In another embodiment, the method comprises the step of assessing the clinical symptoms of the mammal.

In another embodiment, the mammal is a human.

In another embodiment, the test sample is selected from the group consisting of plasma, serum, whole blood, urine, sweat, lymph, faeces, tears, nasal fluid or nasal neuroepithelium, and cerebrospinal fluid.

In another embodiment, the antibody-antigen complexes are determined using an immunoassay or a chemiluminescent assay.

A method of treating and/or preventing a neurological disorder with immune reaction in patient is also provided. The method can comprise:

i) assessing the clinical symptoms corresponding to a neurological disorder with immune reaction in the patient;
ii) detecting the presence or absence of autoantibodies to an epitope of at least one Trib2 protein or an antigenic determinant thereof in a test sample obtained from the patient according to the method of diagnosing a neurological disorder with immune reaction; and
iii) administering to a patient in need thereof a compound inactivating the autoantibody.

In one embodiment, the method of treating and/or preventing a neurological disorder with immune reaction further comprises the step of detecting orexin A protein and/or orexin B protein in a test sample from said mammal.

In another embodiment of the method of treating and/or preventing a neurological disorder with immune reaction, the compound that inactivates the autoantibody is selected from the group consisting of beta interferons, interleukin-12, corticosteroids, plasmaphoresis, and intravenous immunoglobulines such as an immunoglobuline G, or a combination of said compounds, or pharmaceutical compositions.

An assay kit for diagnosing a neurological disorder with immune reaction in a mammal is also provided. The assay kit can comprise i) at least one epitope of at least one Trib2 protein or an antigenic determinant thereof; ii) reagents for the constitution of the medium appropriate for carrying out the autoantibody-epitope reaction; and iii) the reagents making possible the detection of the complex formed.

A method of monitoring treatment in a patient suffering from a neurological disorder with immune reaction, wherein the patient is treated by administration of a compound or a pharmaceutical composition is also provided. The method can comprise the steps of:

(a) determining the concentration or amount in a first test sample from a patient of one or more autoantibodies reactive with Trib2 protein, or an antigenic determinant thereof;

(b) treating the patient with one or more pharmaceutical compositions for a period of time;

(c) determining the concentration or amount in a second or subsequent test sample obtained from the patient following treatment in step (b) of one or more autoantibodies reactive with Trib2 protein, or an antigenic determinant thereof; and

(d) comparing the concentration or amount of one or more autoantibodies reactive with Trib2 protein or an antigenic determinant thereof determined in step (c) with the concentration or amount of one or more autoantibodies reactive with Trib2 protein or an antigenic determinant thereof determined in step (a), wherein if the concentration or amount determined in step (c) is unchanged or is unfavorable when compared to the concentration or amount of one or more autoantibodies reactive with Trib2 protein or an antigenic determinant thereof determined in step (a), then the disorder in the subject is determined to have continued, progressed or worsened, and the subject should be treated with a higher concentration of the one or more pharmaceutical compositions administered to the subject in step (b) or the subject should be treated with one or more pharmaceutical compositions that are different then the one or more pharmaceutical compositions administered to the subject in step (b), further wherein, if the concentration or amount of one or more autoantibodies reactive with Trib2 protein or an antigenic determinant thereof determined in step (c) is favorable when compared to the concentration or amount of one or more autoantibodies reactive with Trib2 protein or an antigenic determinant thereof determined in step (a), then the disorder in the subject is determined to have discontinued, regressed or improved, and the subject should continue to be administered the one or pharmaceutical compositions of step (b).

A method of monitoring treatment in a patient suffering from a neurological disorder with immune reaction and treated by administration of a compound or a pharmaceutical composition is also provided. In one embodiment, the method comprises the steps of:

(a) determining the concentration or amount of orexin A protein and/or orexin B protein in said first, or another, test sample from a patient;

(b) treating the patient with one or more pharmaceutical compositions for a period of time;

(c) determining the concentration or amount of orexin A protein and/or orexin B protein in said second, or another, test sample from a patient; and (d) comparing the concentration or amount of orexin A protein and/or orexin B protein determined in step (c) with the concentration or amount of orexin A protein and/or orexin B protein determined in step (a), wherein if the concentration or amount determined in step (c) is unchanged or is unfavorable when compared to the concentration or amount determined in step (a), then the disorder in the subject is determined to have continued, progressed or worsened, and the subject should be treated with a higher concentration of the one or more pharmaceutical compositions administered to the subject in step (b) or the subject should be treated with one or more pharmaceutical compositions that are different then the one or more pharmaceutical compositions administered to the subject in step (b), further wherein, if the concentration or amount of orexin A protein and/or orexin B protein determined in step (c) is favorable when compared to the concentration or amount determined in step (a), then the disorder in the subject is determined to have discontinued, regressed or improved, and the subject should continue to be administered the one or pharmaceutical compositions of step (b).

Other objects and advantages will become apparent to those skilled in the art from a review of the ensuing detailed description, which proceeds with reference to the following illustrative drawings, and the attendant claims.

### DESCRIPTION OF THE FIGURES

FIG. 1. Pabpc 1-hypocretin transgenic mice (A) RT-PCR experiments performed on total RNAs from a transgenic mouse hypothalamus (Tg+) showing the presence of both the construct and hypocretin (Hcrt) transcripts. Four different primer pairs were used, each generating a PCR fragment of the size predicted by its cDNA sequence: 5′ orx/Flag (467 bp), PABP/IRES (333 bp), GFP (611 bp) and orx (189 bp). (B) Quantitative RT-PCR experiments showing similar relative RNA expression levels for Flag-tagged PABP, GFP and orx in transgenic mice hypothalamus. Only the orx transcript was detected in non-transgenic (Tg-) mouse hypothalamus as expected. The normalized relative values (+1 standard deviation) obtained correspond to the mean values from four biological replicates per condition. (C) Western blot analysis demonstrating the presence of the Flag protein in the hypothalamic extract (obtained from three transgenic mice) and its absence in the cortical fraction (from the same three mice). The loading control is a non-specific band generated by the mouse monoclonal anti-Flag M2 antibody. Photomicrographs illustrating the distribution of orx (D,F) and Flag (E,G)—expressing cells on a coronal section from the tuberal hypothalamus. Higher magnifications confirm that orx and Flag are co-expressed in the same neurons. Arrows indicate both single orx or Flag or double orx-Flag staining. Almost all orx neurons were found to express the Flag containing construct. cp: cerebral peduncle, fx: formix, mtt: mammilo-thalamic tract, 3V: third ventricle. Scale bars in D and E 100 μm, in F, G, H 20 μm.

FIG. 2. ELISA determination of anti-Trib2 antibodies in sera. (A) Each dot corresponds to the serum of a single subject. Mean and ±1 standard deviation of each group is reported next to individual values. Dotted horizontal line indicates the mean anti-Trib2 titer in healthy control subjects +2 standard deviations. All values are relative to the optical density of a healthy control subject (=1). P values correspond to independent t-tests between indicated groups. OIND: other inflammatory neurological disorders. (B) Mean±1SD of anti-Trib2 titers at different intervals from the disorder onset. The horizontal plain and dotted lines indicate mean titer ±1SD in normal control subjects. Numbers indicate the number of narcolepsy patients at each interval. Note the sharp titers decrease within the first 2-3 years reaching normal values. From 5 up to 30 years after disorder onset, the titers remain stable but significantly (1SD, p<5×10−5) higher than healthy control subjects (n=42).

FIG. 3. Double immunohistochemistry with hypocretin antibody and a high anti-Trib2 antibody-containing serum of a narcolepsy with cataplexy patient. (A) Coronal section through the mouse hypothalamus stained with anti-hypocretin antibody. (B) The same section stained with the patient's serum. (C) Merged hypocretin-serum staining showing that the vast majority of hypocretin-positive neurons are co-stained by the patient's serum. Higher magnifications (D-F) confirm the double-labeling of hypocretin neurons. Overall, 87% of hypocretin neurons are co-stained by the serum. (G) Another mouse hypothalamic section stained with anti-hypocretin antibody. (H) Immunoreactivity with the same patient's serum as in (B) but after pre-absorption of anti-Trib2 antibodies with excess Trib2 peptide. (I) Merged image showing that the vast majority of hypocretin neurons are only stained with hypocretin-antibody. Only 8% of hypocretin neurons are double-labeled. Higher magnifications (J-L) confirm that serum depletion of anti-Trib2 antibodies results in the absence of double staining Scale bars=20 μm.

FIG. 4. Generation of Pabp1-orexin transgenic mice. A: orexin gene structure within the BAC (RP23-279L23) used for transgene construction. B: the construction including the coding sequence of Flag-Tagged Pabp1 and eGFP, the selection cassette Zeomycine and recombinase recognition sites FRA was PCR amplified with primers corresponding to the immediate 5′ and 3′ region of the orexin gene. C: this construct was recombined with the BAC in EL250 competent cells, before removing the selection cassette (D) and pronucleus injection (E).

FIG. 5. mRNA Immunoprecipitation of orexin and MCH from total brain RNA extracts. To test the specificity of our construct and immunoprecipitation technique, the ratio of orexin to MCH mRNA was compared between pull down and whole brain extracts that showed a 20 fold increased in orexin transcript.

### DETAILED DESCRIPTION OF THE INVENTION

To screen for hypocretin-coexpressed peptides that may be targets of an autoimmune attack in narcolepsy, Applicants have engineered a transgenic mouse model. As shown in the Results part, Tribbles homolog 2 transcript (Trib2) is enriched over 3 fold in hypocretin-producing neurons.

Surprisingly, Applicants found that patients suffering from a neurological disorder with immune reaction, such as narcolepsy, have high titers of circulating autoantibodies against Trib2 as compared to all other groups. Moreover, they showed that serum of a narcolepsy patient showed immunoreactivity with over 86% of hypocretin neurons in the mouse hypothalamus and that preabsorption of the serum with recombinant Trib2 peptide abolished immunorectivity. These results indicate for the first time that Trib2 is an autoantigene in neurological autoimmune disorders such as human narcolepsy and that anti-Trib2 antibodies specifically target hypocretin neurons, ultimately leading to their disappearance and hypocretin deficiency.

High anti-Trib2 titers (>2.5-3 SD) were found in narcolepsy with cataplexy patients exclusively, but Applicants did not find an overall difference between narcolepsy with and without cataplexy. Furthermore, antibody titers correlate both with the severity of cataplexy and sleepiness, suggesting that the two conditions might share a similar pathophysiology.

Although neither Trib2 nor any other peptide was found specific to hypocretin neurons, given the low level of Trib2 expression but its specific enrichment in hypocretin neurons, it may be that in addition to other sporadic neurons, hypocretin neurons are the major target of the autoimmune attack, resulting primarily in narcolepsy symptoms. Nevertheless, other colocalized and/or enriched transcripts such as Igf2 bp2 or Slc12a6, might play some functional role in hypocretinergic neurotransmission, as also suggested for Igfbp3 (18). Anti-Trib2 antibodies seem to predominate in narcolepsy patients with recent disorder onset further suggesting that these antibodies are pathogenic. Although only 14% of all our narcolepsy patients had anti-Trib2 titers over 2SD above controls, 30% of patients with an interval from disorder onset below 1 year had titers >2SD. Anti-Trib2 titers sharply decrease within the first 2-3 years of disorder onset but remains significantly higher than controls up to 30 years, suggesting that narcolepsy might be triggered by an acute autoimmune process and not a recurrent process as in many other autoimmune disorders. Together with HLA and T cell receptor alpha associations, Applicants findings strongly suggest that narcolepsy can be an autoimmune disorder.

Obviously not all narcolepsy patients have high anti-Trib2 titers even close to the disorder onset, suggesting more complex mechanisms leading to the development of the condition. The time-course of a potential autoimmune process targeting hypocretin neurons may vary from patient to patient. Some patients may have a rapid course with major deficit in hypocretin production, resulting in sudden appearance of excessive daytime sleepiness and cataplexy, while others may have a slower course resulting in excessive daytime sleepiness followed, up to several years later, by cataplexy when the number of hypocretin neurons reaches a critical value. It may also be that Trib2 is not the only autoantigen and/or not the most specific one. As in other autoimmune disorders (e.g. type 1 diabetes), several autoantibodies may be involved but not all are detectable in the majority of patients.

Based on the aforementioned results, the Applicants have developed a method of diagnosing a neurological disorder with immune reaction in a mammal comprising the step of detecting, in a test sample from the mammal, autoantibodies to an epitope of at least one Trib2 protein or an antigenic determinant thereof.

Usually, the method of the present invention comprises the steps of a) contacting the test sample with the at least one Trib2 protein or an antigenic determinant thereof; and b) detecting any antibody-antigen complexes formed between the at least one Trib2 protein or an antigenic determinant thereof and antibodies present in the test sample, wherein presence of said complex is indicative of a neurological disorder with immune reaction.

An “antigenic determinant” of the present invention refers to a part of a Trib2 protein, which binds to an antibody-combining site or to a T-cell receptor (TCR). Alternatively, an “antigenic determinant” may be a site on the surface of a Trib2 protein of the present invention to which a single antibody molecule binds. Generally an antigen has several or many different antigenic determinants and reacts with antibodies of many different specificities. Preferably, the antibody is an autoantibody immunospecific to a Trib2 protein.

Antigenic determinants usually consist of chemically active surface groupings of molecules, such as amino acids or sugar side chains, and can have specific three dimensional structural characteristics, as well as specific charge characteristics.

Examples of antigenic determinants comprise whole antigens, immunogenic domains, or epitopes.

The term “epitope” includes any protein determinant capable of specific binding to an immunoglobulin or T-cell receptor.

“Autoantibodies” or “autoantibody” refers to endogenous antibodies that bind to (or react with) an analyte that occurs naturally in the animal in which the antibody is produced. In the context of the present disclosure, the analyte is the Trib2 protein or a antigenic determinant thereof thereof. Use of “at least one” and “one or more” with respect to autoantibodies means at least one or one or more types or populations of autoantibodies, i.e., such as autoantibodies that react with different epitopes or antigenic déterminants on Trib2 protein or antigenic determinant thereofs thereof.

“Trib2 protein” or “Tribbles homolog 2” refers to one of the mammalian homolog of Drosophila tribbles. All tribbles proteins closely resemble serine-threonine kinases, but are believed to be functionally dead as they contain a variant catalytic core and lack the ATP binding site of conventional kinases. The N-terminal region shows the least homology amongst Trib family members and to Drosophila tribbles (Kiss-Toth et al., Cell Signal 18:202-214 (2006)). Drosophila embryos with tribbles loss of function have low viability, with only 14% of mutant flies surviving to adulthood, and loss of homozygotes did appear to occur at a specific developmental stage. Mammalian data for Trib2 is limited, with one study identifying Trib2 as a candidate autoantigen in autoimmune uveitis from patient eye samples (14).

“Neurological disorders with immune reaction” are disorders characterized by an immune system's failure to recognize self. Examples of such neurological disorders include, but are not limited to, the group comprising narcolepsy with or without cataplexy, idiopathic hypersomnia, Kleine Levin syndrome, multiple sclerosis, Parkinson's disease, Alzheimer's disease, Guillain-Barre syndrome, uveitis, Myasthenia gravis, Neuromyotonia, Neuropathic diabetes, Schizophrenia, and other neuroinflammatory disorders. Other disorders where the orexin neurons may be destroyed or critically involved include, but are not limited to, obesity, addiction, secondary hypersomnias due to tumors, head trauma, and stroke. The term “immune recation” is used in its singular form but includes one or more immune reations.

As used herein, the term “test sample” generally refers to a biological material being tested for and/or suspected of containing at least one autoantibody of interest, such as a myeloperoxidase. The test sample may be derived from any biological source, such as, a physiological fluid, including, but not limited to, plasma, serum, whole blood, urine, sweat, lymph, faeces, tears, nasal fluid and nasal neuroepithelium, and cerebrospinal fluid, and other suitable bodily fluids. The test sample may be used directly as obtained from the biological source or following a pretreatment to modify the character of the sample. For example, such pretreatment may include preparing plasma from blood, diluting viscous fluids and so forth. Methods of pretreatment may also involve filtration, precipitation, dilution, distillation, mixing, concentration, inactivation of interfering components, the addition of reagents, lysing, etc. Moreover, it may also be beneficial to modify a solid test sample to form a liquid medium or to release at least one autoantibody.

Examples of mammals include, but are not limited to, dogs, cats, rabbits, mice, rats, goats, sheep, cows, pigs, horses, non-human primates and humans. Preferably the mammal is a human.

As used in this specification, the singular forms “a”, “an”, and “the” include plural references unless the context clearly dictates otherwise. Thus, for example, a reference to “the method” includes one or more methods, and/or steps of the type described herein and/or which will become apparent to those persons skilled in the art upon reading this disclosure and so forth.

Since applicants have shown that titers of autoantibodies against Trib2 correlated with the severity of, for example, cataplexy, the present invention also provides the step of assessing the clinical symptoms of the mammal. The amount of any antibody-antigen complexes formed between the at least one Trib2 protein or an antigenic determinant thereof and antibodies present in the test sample is indicative and directly correlated with the severity of orexin deficiency in the neurological disorder with immune reaction.

After obtaining the test sample from a mammal said the test sample is being assessed for autoantibodies to Trib2 protein (or an antigenic determinant thereof) and a first specific binding partner, wherein the first specific binding partner and any autoantibodies contained in the test sample form a first specific binding partner-autoantibody complex.

Preferably, the first specific binding partner is Trib2 protein or an antigenic determinant thereof.

In one embodiment, a DNA sequence encoding either the Trib2 protein or a antigenic determinant thereof is fused to a tag such as but not limited to GST or His. This fusion protein is usually used to purify and detect antibodies of interest. In a GST gene fusion system, the GST sequence is incorporated into an expression vector alongside the gene sequence (e.g. cDNA) encoding the Trib2 protein or an antigenic determinant thereof. Induction of protein expression from the vector's promoter results in expression of a fusion protein: the Trib2 protein or an antigenic determinant thereof fused to the GST protein. This GST-Trib2 protein (or a antigenic determinant thereof) can then be purified from cells via its high affinity for glutathione. Once expressed and purified, the GST fusion protein containing the Trib2 protein or an antigenic determinant thereof is immobilized on a solid phase.

Preferably, the Trib2 antigenic determinant is either the full length human TRIB2 protein of SEQ ID No1:

(SEQ ID NO: 1)
MNIHRSTPITIARYGRSRNKTQDFEELSSIRSAEPSQSFSPNLGSPSPPE
TPNLSHCVSCIGKYLLLEPLEGDHVFRAVHLHSGEELVCKVFDISCYQES
LAPCFCLSAHSNINQITEIILGETKAYVFFERSYGDMHSFVRTCKKLREE
EAARLFYQIASAVAHCHDGGLVLRDLKLRKFIFKDEERTRVKLESLEDAY
ILRGDDDSLSDKHGCPAYVSPEILNTSGSYSGKAADVWSLGVMLYTMLVG
RYPFHDIEPSSLFSKIRRGQFNIPETLSPKAKCLIRSILRREPSERLTSQ
EILDHPWFSTDFSVSNSAYGAKEVSDQLVPDVNMEENLDPFFN,

its 28 C-terminal amino acids having the following sequence SEQ ID No 2:

| | |
|---|---|
| NSAYGAKEVSDQLVPDVNMEENLDPFFN, | (SEQ ID NO: 2) |

or any fragment of said SEQs ID No 1 or 2.

The order in which the test sample and the first specific binding partner are added to form a mixture is not critical. Preferably, the first specific binding partner is immobilized on a solid phase. The solid phase used in the immunoassay (for the first specific binding partner and, optionally, the second specific binding partner) can be any solid phase known in the art, such as, but not limited to, a magnetic particle, a bead, a test tube, a microtiter plate, a cuvette, a membrane, a scaffolding molecule, a film, a filter paper, a disc and a chip. After the mixture containing the first specific binding partner-autoantibody complex is formed, any unbound autoantibodies are removed from the complex using any technique known in the art. For example, the unbound autoantibodies can be removed by washing.

After any unbound autoantibodies are removed, a second specific binding partner is added to the mixture to form a first specific binding partner-autoantibody-second specific binding partner complex. The second specific binding partner is preferably an anti-human antibody. Moreover, also preferably, the second specific binding partner is labeled with or contains a detectable label.

In terms of the detectable label, any detectable label known in the art can be used. For example, the detectable label can be a radioactive label (such as, e.g., ³H, ¹²⁵I, ³⁵S, ¹⁴C, ³²P, and ³³P), an enzymatic label (such as, e.g., horseradish peroxidase, alkaline peroxidase, glucose 6-phosphate dehydrogenase, and the like), a chemiluminescent label (such as, e.g., acridinium esters, luminal, isoluminol, thioesters, sulfonamides, phenanthridinium esters, and the like), a fluorescence label (such as, e.g., fluorescein (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)), dyes, rhodamine, phycobiliproteins, R-phycoerythrin, quantum dots (e.g., zinc sulfide-capped cadmium selenide), a thermometric label, photosensitizers or an immuno-polymerase chain reaction label. An introduction to labels, labeling procedures and detection of labels is found in Polak and Van Noorden, Introduction to lmmuno cytochemistry, 2nd ed., SpringerVerlag, N.Y. (1997) and in Haugland, Handbook of Fluorescent Probes and Research Chemicals (1996), which is a combined handbook and catalogue published by Molecular Probes, Inc., Eugene, Oreg.

Preferably, the detectable label is selected from dyes, fluorescers, radiolables, enzymes, chemiluminescers, photosensitizers.

Also preferably, the second specific binding partner is an anti-human antibody labeled with biotin.

Usually, the antibody-antigen complexes are determined using an immunoassay, immuno-real time PCR or a chemiluminescent assay.

Examples of immunoassay are selected form the non-limiting group comprising affinity chromatography, Immunoelectrophoresis, radioimmunoassay (RIA), enzyme-linked immunosorbent assays (ELISAs), immunofluorescent assays, Western blotting, and the like.

In another embodiment, immuno-PCR can be used to detect any of the autoantibodies against Trib2 protein or a antigenic determinant thereof. Immuno-PCR is a modification of the conventional ELISA format in which the detecting antibody is labeled with a DNA label, and is applicable to the analysis of biological samples (see, e.g., U.S. Pat. No. 5,665,539 and U.S. Patent Application Publication No. 2005/0239108; Refs 33 & 34, all herein incorporated by reference). The amplification ability of PCR provides large amounts of the DNA label which can be detected by various methods, typically gel electrophoresis with conventional staining (e.g., Sano et al, Science, 258:120-122, 1992). This method can also include the direct conjugation of the DNA label to the antibody and replacement of gel electrophoresis by using labeled primers to generate a PCR product that can be assayed by ELISA or using real time quantitative PCR. In an example of the real-time PCR method, PCR is used to amplify DNA in a sample in the presence of a nonextendable dual labeled fluorogenic hybridization probe. One fluorescent dye serves as a reporter and its emission spectra is quenched by the second fluorescent dye. The method uses the 5′ nuclease activity of Taq polymerase to cleave a hybridization probe during the extension phase of PCR. The nuclease degradation of the hybridization probe releases the quenching of the reporter dye resulting in an increase in peak emission from the reporter. The reactions are monitored in real time.

An example of commercially available immuno-PCR kit is the Imperacer™ Kit developed by Chimera Biotec.

Immuno real time PCR (31), quantitative real-time immuno-PCR (30) and immuno-detection amplified by T7 RNA polymerase (IDAT, 32) are also techniques envisioned to be used to detect any of the autoantibodies against Trib2 protein or a antigenic determinant thereof.

Chemiluminescent signals generated can be detected using routine techniques known to those skilled in the art.

Since the levels of autoantibodies against Trib2 protein or an antigenic determinant thereof usually correlate with the extent of orexin neuronal loss, the present invention also contemplates the simultaneous or previous detection of orexin A protein and/or orexin B protein, and autoantibodies against Trib2 protein or an antigenic determinant thereof, for a quantitative diagnosis of autoimmunity and orexin deficiency. This orexin A and/or orexin B level(s) detection is usually done through similar techniques as those used for the detection of Trib2 protein or an antigenic determinant thereof.

Orexin deficiency is so far detected mainly by Radio-immunoassay with limited resolution and orexin deficiency is defined by a level lower than the third of normal healthy subjects. Therefore the preferred method of detection of orexin A and/or orexin B is immuno-PCR.

“Orexin A protein” and/or “orexin B protein” refer to neuropeptide hormones derived from proteolytic processing of the same orexin precursor protein. They are released from the hypothalamus and are involved in the regulation of sleeping patterns, feeding, and metabolism. Orexins A and B are less than 50% amino acid identical. Orexin A is a ligand for two 7-transmembrane orexin receptors, OX1R and OX2R, while orexin B preferentially binds OX2R.

Usually, orexin A protein and/or orexin B protein is/are detected in the same test sample as that used for the detection of autoantibody.

Alternatively, the test sample is different from that used for the detection of autoantibody.

Applicants have reported several cases of narcolepsy treated with intravenous immunoglobulins (IVIg) soon after the first symptoms with unexpected positive results, suggesting that the autoimmune process may be counteracted if treated early (21, 22). Most recently they have reported a narcolepsy patient with hypocretin deficiency in whom the CSF hypocretin level normalized after IVIg treatment (23). The present findings strengthen the autoimmune hypothesis and suggest that early-diagnosed patients with high anti-trib2 titers might benefit from immunotherapy.

Therefore, in another embodiment, the present invention also provides a method of treating and/or preventing a neurological disorder with immune reaction in patient comprising

i) assessing the clinical symptoms corresponding to a neurological disorder with immune reaction in the patient suggestive of orexin deficiency,
ii) detecting the presence or absence of autoantibodies to an epitope of at least one Trib2 protein or an antigenic determinant thereof in a test sample obtained from the patient according to the method of the present invention,
iii) detecting the level of orexin A protein and/or orexin B protein to correlate autoantibody titers and orexin levels,
iii) administering to a patient in need thereof a compound inactivating the autoantibody.

Preferably, the compound, or pharmaceutical composition, which inactivates the autoantibody is selected from the group comprising but not limited to beta interferons, interleukin-12, corticosteroids, plasmaphoresis, and intravenous immunoglobulines or a combination of said compounds, or pharmaceutical compositions.

Most preferably, the compound, or pharmaceutical composition is an immunoglobuline G preparation or pharmaceutical composition.

In still yet another embodiment, the present disclosure relates to a method of monitoring treatment in a patient, e.g., treatment of a patient for neurological disorder with immune reaction by administration of a compound, or a pharmaceutical composition. The method comprises the steps of:

(a) determining the concentration or amount in a first test sample from a patient of one or more autoantibodies reactive with Trib2 protein, or an antigenic determinant thereof;

(b) determining the concentration or amount of orexin A protein and/or orexin B protein in said first, or another, test sample from a patient,

(c) treating the patient with one or more pharmaceutical compositions for a period of time;

(d) determining the concentration or amount in a second or subsequent test sample obtained from the patient following treatment in step (b) of one or more autoantibodies reactive with Trib2 protein, or an antigenic determinant thereof; and

(e) determining the concentration or amount of orexin A protein and/or orexin B protein in said second, or another, test sample from a patient,

(f) comparing the concentration or amount of one or more autoantibodies reactive with Trib2 protein or an antigenic determinant thereof determined in step (d) with the concentration or amount of one or more autoantibodies reactive with Trib2 protein or an antigenic determinant thereof determined in step (a), wherein if the concentration or amount determined in step (d) is unchanged or is unfavorable when compared to the concentration or amount of one or more autoantibodies reactive with Trib2 protein or an antigenic determinant thereof determined in step (a), then the disorder in the subject is determined to have continued, progressed or worsened, and the subject should be treated with a higher concentration of the one or more pharmaceutical compositions administered to the subject in step (c) or the subject should be treated with one or more pharmaceutical compositions that are different then the one or more pharmaceutical compositions administered to the subject in step (c), further wherein, if the concentration or amount of one or more autoantibodies reactive with Trib2 protein or an antigenic determinant thereof determined in step (d) is favorable when compared to the concentration or amount of one or more autoantibodies reactive with Trib2 protein or an antigenic determinant thereof determined in step (a), then the disorder in the subject is determined to have discontinued, regressed or improved, and the subject should continue to be administered the one or pharmaceutical compositions of step (c).

Alternatively, it is also envisioned to compare the concentration or amount of orexin A protein and/or orexin B protein determined in step (e) with the concentration or amount of orexin A protein and/or orexin B protein determined in step (b), wherein if the concentration or amount determined in step (e) is unchanged or is unfavorable when compared to the concentration or amount determined in step (b), then the disorder in the subject is determined to have continued, progressed or worsened, and the subject should be treated with a higher concentration of the one or more pharmaceutical compositions administered to the subject in step (e) or the subject should be treated with one or more pharmaceutical compositions that are different then the one or more pharmaceutical compositions administered to the subject in step (e), further wherein, if the concentration or amount of orexin A protein and/or orexin B protein determined in step (e) is favorable when compared to the concentration or amount determined in step (b), then the disorder in the subject is determined to have discontinued, regressed or improved, and the subject should continue to be administered the one or pharmaceutical compositions of step (e).

Moreover, the present disclosure also relates to methods of determining whether a subject predisposed to or suffering from a neurological disorder with immune reaction (e.g., narcolepsy) will benefit from treatment with a drug, and the response of a subject receiving treatment by monitoring one or more autoantibodies reactive with Trib2 protein or an antigenic determinant thereof.

Another concern of the present invention is to provide an assay kit for diagnosing a neurological disorder with immune reaction in a mammal comprising at least one epitope of at least one Trib2 protein or an antigenic determinant thereof, reagents for the constitution of the medium appropriate for carrying out the autoantibody-epitope reaction and the reagents making possible the detection of the complex formed.

Alternatively, the assay kit for diagnosing a neurological disorder with immune reaction in a mammal further comprises reagents for detecting orexin A protein and/or orexin B protein in said, or another, test sample.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practicing the present invention and are not intended to limit the scope of the invention.

### Examples

### Methods

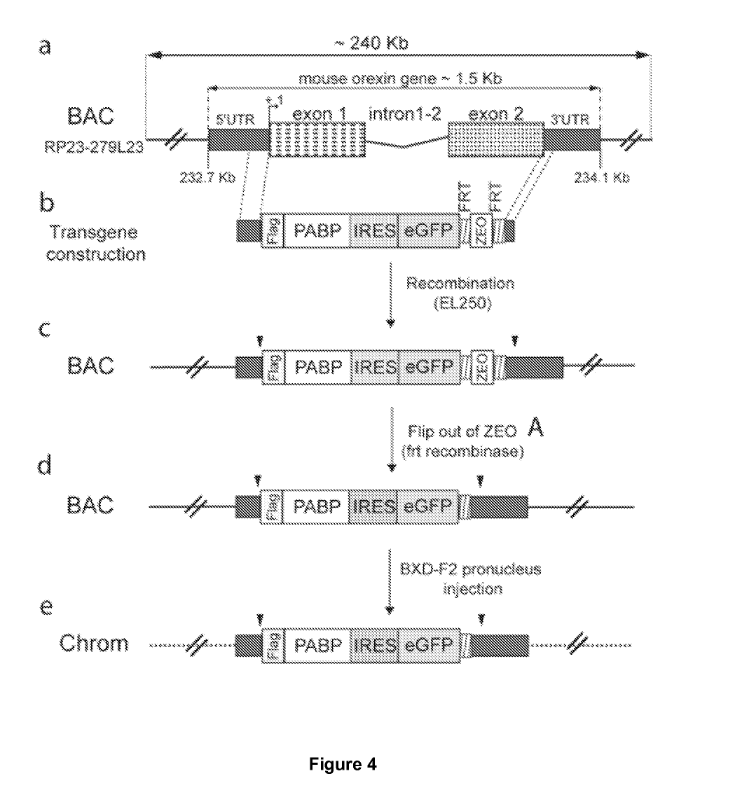

Transgenic mice production, mRNA tagging, and immunoprecipitation. BAC-based Flag-tagged PABP-hypocretin transgenic mice were generated following a procedure (FIG. 4) similar to that described for the generation of Horner1a-PABP transgenic mice (15). The study protocol was approved by the Lausanne Veterinary Office.
Our mRNA tagging process was modified from two previously described procedures in Caenorhabditis elegans and Drosophila (24, 25) and was demonstrated by us to successfully enrich Horner/a-specific mRNAs in Homerla-PABP transgenic mice (15).
RT-PCR and Real-time RT-PCR experiments. RT-PCR genotyping of transgenic mice were performed with primers specific for the construction (PABP/IRES forward 5′-AAGCCATGCACCCTTCTCTTGC-3′ (SEQ ID NO:3), reverse 5′-CCTTATTCCAAGCGGCTTCGG-3′ (SEQ ID NO:4); GFP forward 5′-GAGCTGGACGGCGACGTAAACG-3′ (SEQ ID NO:5), reverse 5′-AGGACCATGTGATCGCGCTTCTC-3′ (SEQ ID NO:6)). Quantitative RT-PCR experiments to detect endogenous hypocretin, our construct, and candidate genes were performed by TaqMan Universal PCR Master Mix (Applied Biosystems) in 384-well plates in an ABI PRISM 7900 detection system (Applied Biosystems) following standard procedures Gene-specific primers and TaqMan probes were as follows: Hypocretin TaqMan gene expression assay (Applied Biosystems), Pmch forward, 5′-AGAGATTTTGACATGCTCAGGTGTA-3′ (SEQ ID NO:7), reverse, 5′-GCGGACCAGCAGGTATCAGA-3′ (SEQ ID NO:8), probe, 5′-CTGGGAAGAGTCTACCGACCATGTTGGC-3′ (SEQ ID NO:9), flag-Pabpc1 forward, 5′-TAAAGATCATGACATCGATTACAAGGA-3′ (SEQ ID NO:10), reverse, 5′-AGGTCCCCCACGTACAGAGA-3′ (SEQ ID NO:11), probe, 5′-ACGATGACAAGCTCGACAT-3′ (SEQ ID NO:12), GFP forward, 5′-CTGCTGCCCGACAACCAC-3′ (SEQ ID NO:13), reverse, 5′-ACCATGTGATCGCGCTTCTC-3′ (SEQ ID NO:14), probe, 5′-CCAGTCCGCCCTGAGCAAAGACC-3′ (SEQ ID NO:15), all from Eurogentec.
Western Blotting analysis. Three transgenic mice were rapidly sacrificed and coronal brain slices (300 μm-thick) were cut on a vibratome in artificial cerebrospinal fluid. Slices containing hypocretin neurons were selected and micro-dissected with a razor blade in order to keep only the hypothalamic region. All hypothalamic-dissected regions were pooled together and protein extract prepared with RIPA lysis buffer on ice. Cortex punches from each slice were also collected, pooled together and similarly treated as a control extract. Protein concentration was calculated by using Bradford assay and 32 μg of each fraction were separated on a SDS-PAGE and transferred to a Nitrocellulose membrane. After one hour blocking in Tween/Tris-buffered saline and 5% non-fat milk, membranes were incubated overnight with the monoclonal mouse Anti-Flag® M2 primary antibody (Sigma-Aldrich) diluted 1/300 followed by one hour with the secondary horseradish peroxidase-conjugated goat anti-mouse antibody diluted 1/6000 and revealed using ECL reagents and Hyperfilm ECL.
Double Immunohistochemistry for hypocretin and Flag. Paraformaldehyde-fixed brains were serially cut into 35 μm coronal floating slices on a cryostat. Sections, taken at 140 μm intervals throughout the whole hypothalamus, were processed for a double immunofluorescence procedure. The Flag staining was first revealed with the mouse monoclonal Anti-Flag® M2 antibody diluted 1/500 followed by two hours with the Alexa Fluor® 488 conjugated goat anti-mouse IgG1 secondary antibody diluted 1/1000. After rinsing in PBS, the hypocretin labeling was performed with a rabbit anti-hypocretin A serum diluted 1/2000 followed by Alexa Fluor® 555 conjugated goat anti-rabbit IgG (H+L) secondary antibody diluted 1/1000. Sections were observed with a Zeiss Axioskop 2 microscope (filter sets 10, 15) equipped with an axiocam color CCD camera. Images were recorded on computer through the AxioVision™ software. A confirmation survey on a confocal microscope was also performed.
Microarray experiments. Targets were prepared from either 5 ng of immunoprecipitated (IP) mRNAs or 5 ng of whole brain (WB) total RNAs using the NuGen WT-Ovation Pico RNA amplification system. The generated cDNAs (5 μg) were fragmented, biotinylated and hybridized using the NuGen FL-Ovation cDNA Biotin Module V2 following the manufacturer's instructions. The resulting targets were hybridized to Affymetrix GeneChip Mouse Genome 230 2.0 arrays. All statistical analysis were performed using the free high-level interpreted statistical language R (R Core, 2004, http://www.R-project.org) and various Bioconductor packages (http://www.Bioconductor.org). Hybridization quality was assessed using Bioconductor “affy” and “affyPLM” packages (26, 27). Because of the differences between expression profiles of IP and WB samples, normalized expression signals were calculated in Affymetrix Expression Console using MAS 5.0, which simply scales the overall expression signal to the same intensity. Differential hybridized features were identified from all probe sets using Bioconductor package “limma” on log 2 transformed MAS 5.0 normalized values (28). P values were adjusted for multiple testing with Benjamini and Hochberg's method to control the false discovery rate (FDR). Probe sets showing at least 3-fold change and a FDR <0.05 were considered significant. Data are available at the Gene Expression Omnibus database (http://www.ncbi.nlm nih gov/geo, accession number GSE17617).
Subjects. Narcolepsy with cataplexy, narcolepsy without cataplexy, and idiopathic hypersomnia patients were included based on diagnostic criteria of the International Classification of Sleep Disorders (ICSD-2). The frequency of cataplexy was assessed by a scale from 1 to 5 reporting rare to very frequent cataplexy attacks (29). Healthy controls were recruited in affiliated sleep centers and had full clinical and in more than half polysomnography investigations. Affiliated neurology departments recruited multiple sclerosis and other inflammatory neurological patients. All participants gave written informed consent and the ethics committee of the Faculty of Biology and Medicine of the University of Lausanne approved the study protocol.
ELISA. A GST fusion protein containing 28 C-terminal amino acids (NSAYGAKEVSDQLVPDVNMEENLDPFFN) (SEQ ID NO: 2) of Trib2 was expressed and purified (14). The vector containing the recombinant peptide was a generous gift of Wei Li, University of Miami Miller School of Medicine, Miami, USA. Anti-Trib2 antibody quantifications in narcolepsy patients and controls were determined using ELISA standard procedures. Ten mg of GST-TRIB2 partial fusion protein were deposited in 96-well ELISA plates and incubated overnight at 4° C. Wells were washed and blocked one hour at room temperature (RT) with 1% BSA. Hundred ml of diluted serum ( 1/100) or 50 ml of CSF were applied in triplicates for 1 h at RT. Anti-Human IGG-biotin (diluted 1/10000) and Streptavidin-HRP (diluted 1/10000) were then consecutively incubated one hour at RT. Colorimetric assay was performed, using O-phenylenediamine diluted in citrate-phosphate buffer. H₂O₂ 30% was added just before use. After 10 minutes, reaction was stopped with 3N HC1 and absorbance was read at 492 nm For normalization and interplate calibration, 3 serum samples were repetitively quantified on each plate (intra-assay coefficient of variation <6%, inter-assay coefficient of variation <15%). At least 10 sera were tested both against GST-18aa Trib2 or GST-full-length Trib2 with virtually identical results, strongly suggesting that the partial peptide contains an epitope recognized by auto-antibodies.
Double Immunohistochemistry for hypocretin and narcolepsy serum. Immunochemistry was performed on fixed floating 35 μm mouse brain coronal sections. Sections were incubated 24h at RT with a high anti-Trib2 antibody-containing serum of a narcolepsy with cataplexy patient (dilution at ½). They were rinsed in PBS and incubated 2 h at room temperature with an anti-human IgG-biotin antibody (dilution at 1/1000) followed by 2 h of incubation with Cy2-conjugated streptavidin (dilution at 1/1000). A double immunostaining was performed to reveal hypocretin neurons with a rabbit anti-hypocretin A serum (dilution at 1/1000) followed by Alexa Fluor® 555 conjugated anti-rabbit IgG secondary antibody (dilution at 1/1000). To demonstrate specificity of Trib2 labeling with narcolepsy serum, the same procedure was repeated with the narcolepsy serum preabsorbed during 2 h with 18-C terminal amino acids of Trib2 at the concentration of 4 μg/ml.
Statistics. Microarrray gene expression analysis used standard statistical methods explained above. Antibody titers were compared by two-tailed Student's t-test. Other tests included Fisher's exact test, c², Spearman rank correlation, and Pearson's correlation and p values below 0.05 were considered as significant.

### Results

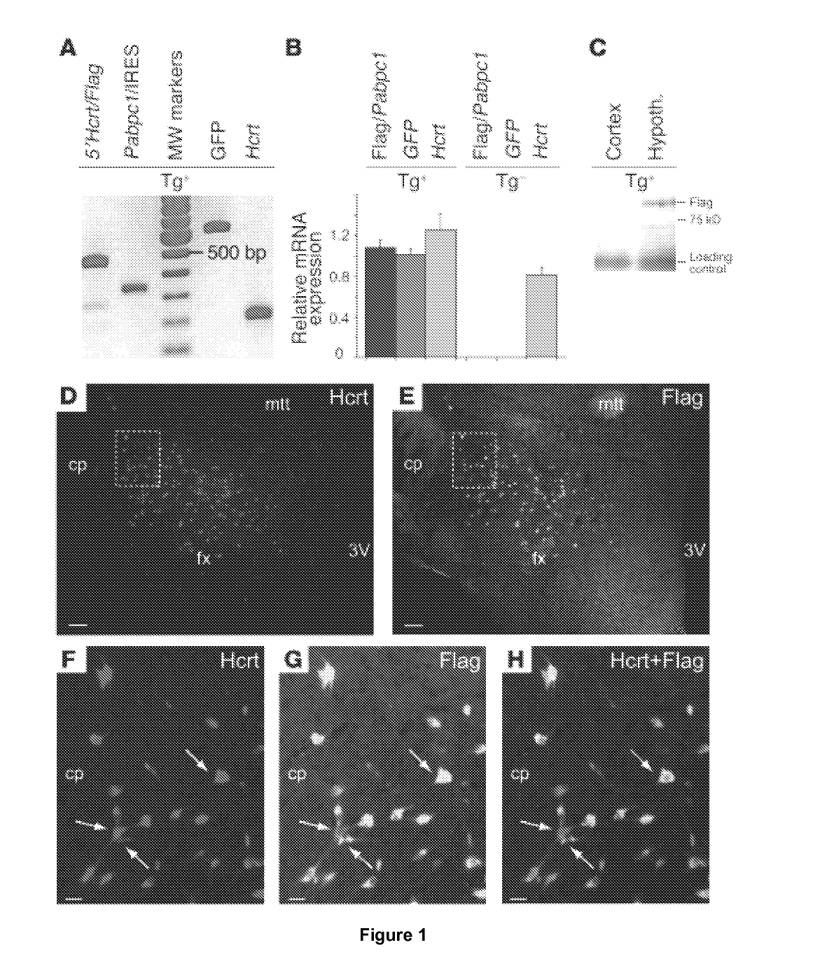

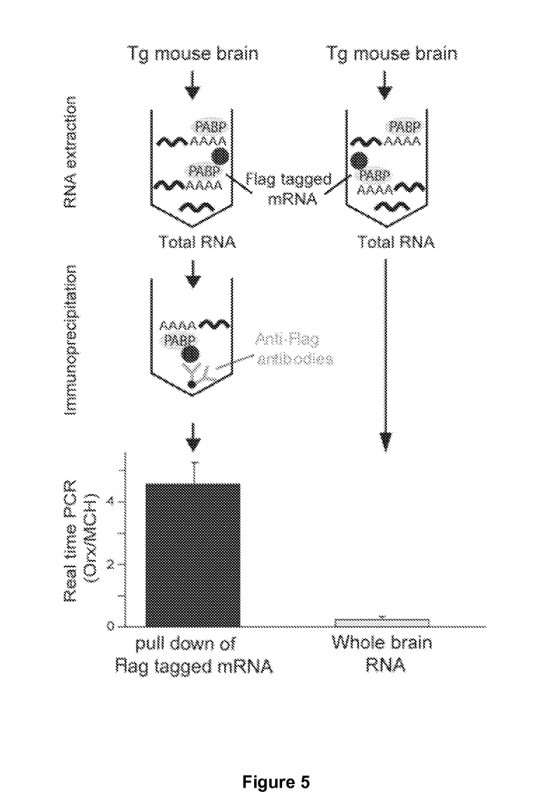

mRNA Tagging and Identification from Hypocretin Producing Neurons
To identify proteins co-expressed with hypocretin, we generated transgenic mice to profile gene expression in hypocretin neurons. In a BAC-based construct we replaced the hypocretin coding sequence by a Flag-tagged poly(A) binding protein (Pabpc1) cDNA sequence (FIG. 4 and Methods). This construct takes advantage of the ability of Pabpc1 to bind the poly(A) tails of mRNAs in vivo. Thus only mRNAs from hypocretin cells are expected to cross-link with the Flag-tagged Pabpc1 which can then be captured by co-immunoprecipitating with a specific anti-flag monoclonal antibody (15). Two transgenic lines were obtained and one was fully characterized as follows. RT-PCR experiments from hypothalamic total RNA extracts, detected the presence of a Flag, a Pabpc1, and an eGFP fragment in transgenic mice (FIG. 1A). Similar relative expression levels of Flag, eGFP, and hypocretin mRNAs were found by real-time RT-PCR in transgenic hypothalami whereas only hypocretin mRNA was detected in non-transgenic mice (FIG. 1B). A monoclonal mouse anti-Flag antibody detected presence of the Flag in mouse hypothalamic protein but not in cortical extracts (FIG. 1C). The specific cellular expression of the Flag-Pabpc1 construct was also demonstrated by double immunofluorescence. All Flag-expressing cells were hypocretin-positive and all hypocretin-expressing neurons were Flag-positive (FIG. 1D-H).
Melanin-concentrating hormone (Pmch) and hypocretin neurons form two distinct but partially overlapping populations in the hypothalamus. To assess mRNA tagging efficacy we compared the ratio of hypocretin mRNA level recovered to that of Pmch in transgenic immunoprecipitated (IP) samples and in transgenic whole brain RNA samples (FIG. 5). Consistent with our expectations, hypocretin mRNA expression was enriched 20-fold after immunoprecipitation. Applying this protocol to non-transgenic mouse brains did not result in hypocretin enrichment.
Comparison between IP-mRNA and total brain mRNA (n=9) in transgenic mice using Affymetrix Mouse Genome Array indicated that 1721 probe sets were enriched at least 3-fold in IP samples (False Discovery Rate: FDR <5%; data not shown). Fold enrichment ranged between 3 and 107. Many of the highly enriched transcripts turned out to be unannotated or matched several genomic localizations. However, probe set 1447461_at was enriched 30-fold and map to an expressed sequence (LOC100048678) with high homology with Spindlins, which are meiotic spindle-binding proteins involved in cell cycle. Over-expression of Spindlinl (Spin1) was recently found to induce cellular senescence, multi-nucleation, and apoptosis (16). Another highly enriched probe set (1420300_at; 16.5-fold) indicated GABA-A2 receptor subunit gene (Gabra2). Although Gabra2 mRNA has been detected in hypocretin-producing neurons, Gabra2 protein was reported not to be expressed (17).

A recent study investigated mRNA expression in the hypothalamic hypocretin region in mice and in post-mortem human narcolepsy samples and identified insulin-like growth factor binding protein 3 (Igfbp3) as a potential co-localized protein (18). In the present study, Igfbp3 was unchanged or slightly depleted from IP-mRNA samples (probe: 1423062_at, fold-change: −1.2, FDR=0.02). Of the two peptides reported to be co-localized with hypocretin, Narp transcript was indeed slightly enriched in IP samples (probe: 1420720_at, fold-change: 2.15, FDR <5.10⁻⁶) while prodynorphin was not (probe 1416266_at, fold change=−1.1, FDR=0.48).

To identify the most specific genes expressed in hypocretin-producing neurons, we selected those probe sets among the 1721 enriched at least 3-fold, that were called present in at least 7 microarrays hybridized with IP-mRNA and called absent in at least 7 hybridized with total brain RNA. This analysis yielded only 23 probe sets (data not shown). None of these 23 transcripts were reported to be specifically expressed in hypocretin neurons based on the mouse brain atlas (Allen Institute for Brain Science; http://mouse.brain-map.org/). Nevertheless among these genes, several are of functional importance and might be involved in abnormal functioning of hypocretin neurons in narcolepsy. Four of such candidate genes (Igf2 bp2, Slc12a6, Spin1, and Trib2) were tested and, except for Spin1, confirmed by quantitative PCR. Trib2 was the only gene found as an autoantigen in an autoimmune disorder, uveitis (14). Intermediate uveitis was found to be associated with HLA-DR15 and narcolepsy in a patient (19). Also one of our narcolepsy patients was affected with uveitis. To explore this further we developed an ELISA assay to detect anti-Trib2 autoantibodies in sera and CSF of narcolepsy patients.

### ELISA Assay Detects Trib2 Autoantibodies in Narcolepsy Patients

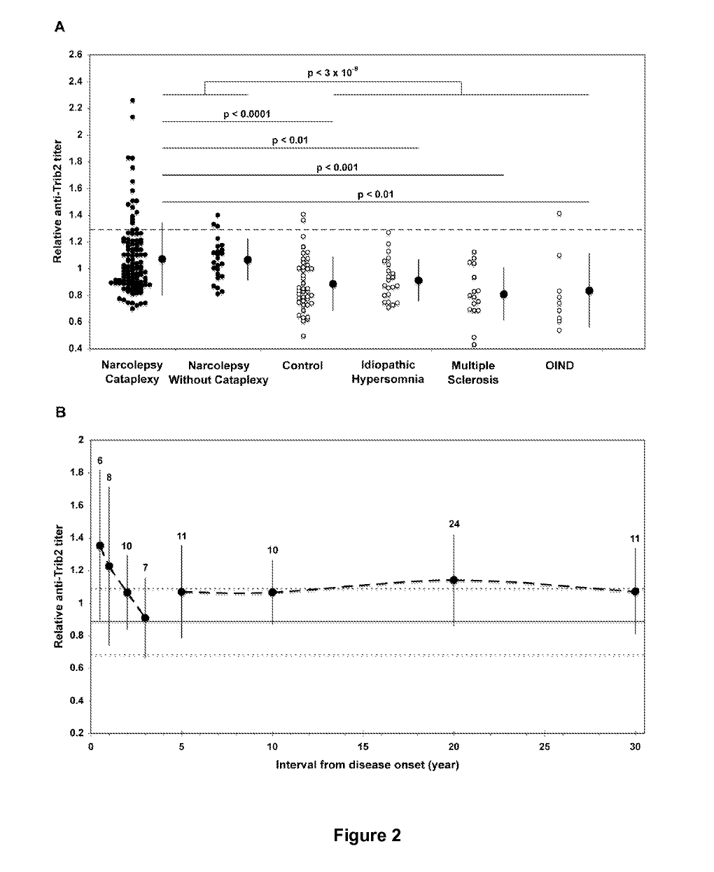

A gluthatione S-transferase-tagged antigenic determinant containing the last 28 amino-acids of Trib2 (14) was used as a target and all 96-well assays were run with serum of a normal subject for normalization. Sera from 119 narcolepsy with cataplexy patients (HLA-DQB1*0602 positive) were run at 1/100 dilution against sera of 24 narcolepsy without cataplexy, 42 normal subjects, 23 idiopathic hypersomnia, 16 multiple sclerosis, and 9 other neurological inflammatory disorders. Relative anti-Trib2 antibody titers were significantly increased in narcolepsy with cataplexy as compared to all other groups (FIG. 2A) except for narcolepsy without cataplexy. When contrasting narcolepsy patients to all others, the relative anti-Trib2 titers were highly significantly increased (p<3×10⁻⁹). Fifty-six out of 143 narcolepsy patients (39%) had titers higher than the mean titer of the controls (>1 standard deviation, SD) and 20 (14%) had titers more than 2 SD above the mean titer of healthy controls. Even with the restrictive 2-SD criterion more narcolepsy patients were positive for anti-Trib2 antibodies than all other groups (20 narcolepsy with (n=17) and without cataplexy (n=3) versus 3 in healthy controls (n=2) and in ONID (n=1), Fisher's exact test, p<0.03). Anti-Trib2 titers of HLA DQB1*0602 positive and negative subjects were not significantly different. In addition to all narcolepsy with cataplexy patients who were DQB1*0602 positive, HLA typing was available in 27 out of 42 controls (64%), 18 out of 23 idiopathic hypersomnia (78%), and 19 out of 24 narcolepsy without cataplexy (79%) and overall 13 (15%) were HLA-DQB1*0602 positive (4 controls, 4 idiopathic hypersomnia, and 5 narcolepsy without cataplexy patients) but none except one narcolepsy without cataplexy had anti-trib2 higher than 2SD above the mean of the healthy control group, strongly suggesting that high anti-Trib2 titers are specific to narcolepsy. In 96 narcolepsy with cataplexy patients the delay between the disorder onset and the time of serum withdrawal could be precisely established. Although we did not find a correlation between anti-Trib2 titers and the duration of disorder, amongst the 17 high anti-Trib2 titer (>2 SD) narcolepsy with cataplexy patients, 5 (29%) were sampled within the first year of disorder onset against 9 (11%) out of the remaining 79 with lower titers (c²=3.65, p<0.06). Anti-Trib2 titers sharply decreased within the first 2-3 years after disorder onset (FIG. 2B) but remained 1SD above the mean titer of healthy controls even 30 years after disorder onset (p<5×10⁻⁵). In 10 out of the 17 patients with >25D titers, complete clinical and laboratory data were available. Anti-Trib2 titers were significantly and positively correlated with the frequency of cataplexy (Spearman rank correlation r=0.74, p<0.02) and tended to be correlated with the severity of sleepiness (Epworth sleepiness scale, r=0.67, p<0.06). CSF samples were available in a limited number of patients (26 narcolepsy with cataplexy, 6 narcolepsy without cataplexy, 4 idiopathic hypersomnia, and 15 multiple sclerosis). Anti-Trib2 antibody could be detected at low levels without a significant difference among groups. Nevertheless, in 18 narcolepsy with cataplexy patients, paired serum-CSF samples could be assessed and a positive correlation was found (r=0.54, p<0.03).

### Trib2-Autoantibodies Target Hypocretin Neurons

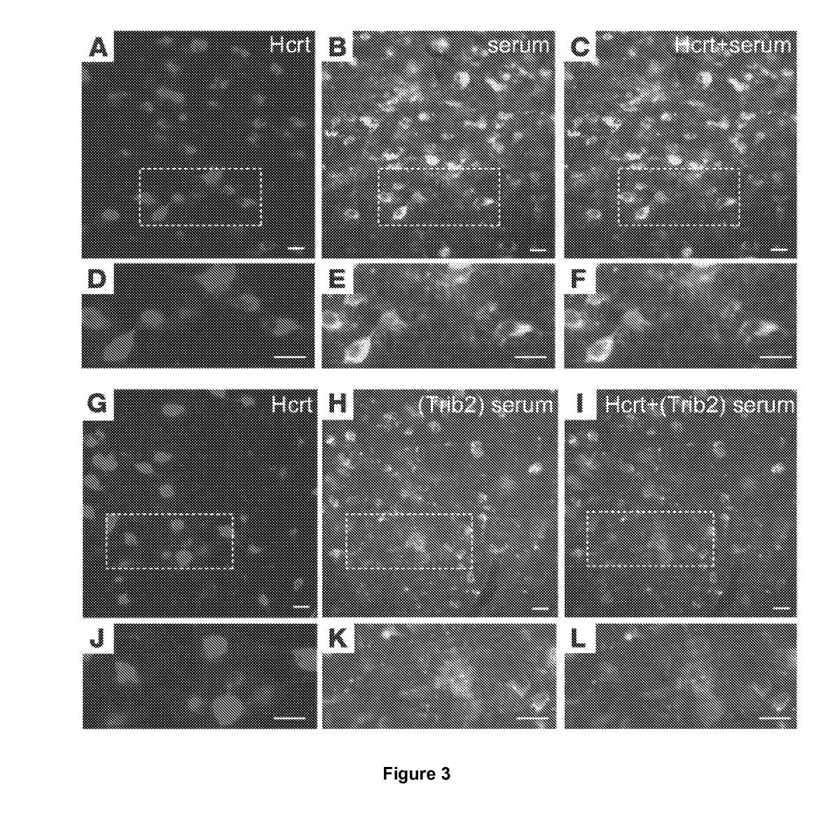

To show that hypocretin-producing neurons are a target of anti-Trib2 antibodies, mouse hypothalamic sections were double-stained with a serum of a narcolepsy with cataplexy patient (age 8, serum available 10 months after disorder onset) with high anti-Trib2 titer and an anti-hypocretin antibody Immunoreactivity against the patient's serum was found throughout the hypothalamus but more specifically over 86% of hypocretin neurons were double-labelled (FIG. 3A-F). To confirm the specificity of the immunoreactivity with hypocretin neurons, the same serum was depleted of anti-Trib2 antibodies by pre-incubation with excess 28 amino-acid Trib2 peptide. Depleted serum showed dramatic decrease (less than 8%) in hypocretin neurons' staining (FIG. 3G-I) confirming the presence of anti-Trib2 autoantibodies targeting hypocretin neurons in the patient's serum. The same experiments with a healthy control serum or the serum from our narcolepsy with uveitis (interval from disorder onset >15 years and anti-trib2 titer within the normal range), did not show any specific staining (between 50 to 60% of hypocretin neurons showed non-specific staining before and after pre-adsorption, respectively).

### Immuno-PCR Assay to Detect Anti-Trib2 Antibodies in Sera.

167 narcolepsy with cataplexy, 29 narcolepsy without cataplexy and 46 control subjects have been tested. Inter-assay variability was 4.2% and Intra-assay variability 2.2% indicating a highly reproducible assay. The mean and standard deviation of anti-Trib2 titers were calculated in control subjects and any titer higher than 2 SD was considered as positive. Two out of 46 control subjects (4.25%) were positive against 42 out of 196 patients (21.43%; p=0.01). The sera in 24 out of the 42 positive patients (57%) were obtained within the first year after the disease onset, confirming that anti-Trib2 titers are much higher if tested close to the disease onset.

### REFERENCES

1. Dauvilliers, Y., Arnulf, I., and Mignot, E. 2007. Narcolepsy with cataplexy. Lancet 369:499-511.
2. Thannickal, T. C., Moore, R. Y., Nienhuis, R., Ramanathan, L., Gulyani, S., Aldrich, M., Cornford, M., and Siegel, J. M. 2000. Reduced number of hypocretin neurons in human narcolepsy. Neuron 27:469-474.
3. Peyron, C., Faraco, J., Rogers, W., Ripley, B., Overeem, S., Charnay, Y., Nevsimalova, S., Aldrich, M., Reynolds, D., Albin, R., et al. 2000. A mutation in a case of early onset narcolepsy and a generalized absence of hypocretin peptides in human narcoleptic brains. Nat Med 6:991-997.
4. Willie, J. T., Chemelli, R. M., Sinton, C. M., Tokita, S., Williams, S. C., Kisanuki, Y. Y., Marcus, J. N., Lee, C., Elmquist, J. K., Kohlmeier, K. A., et al. 2003. Distinct narcolepsy syndromes in Orexin receptor-2 and Orexin null mice: molecular genetic dissection of Non-REM and REM sleep regulatory processes. Neuron 38:715-730.
5. Hara, J., Beuckmann, C. T., Nambu, T., Willie, J. T., Chemelli, R. M., Sinton, C. M., Sugiyama, F., Yagami, K., Goto, K., Yanagisawa, M., et al. 2001. Genetic ablation of orexin neurons in mice results in narcolepsy, hypophagia, and obesity. Neuron 30:345-354.
6. Nishino, S., Ripley, B., Overeem, S., Lammers, G. J., and Mignot, E. 2000. Hypocretin (orexin) deficiency in human narcolepsy. Lancet 355:39-40.
7. Mignot, E., Lammers, G. J., Ripley, B., Okun, M., Nevsimalova, S., Overeem, S., Vankova, J., Black, J., Harsh, J., Bassetti, C., et al. 2002. The role of cerebrospinal fluid hypocretin measurement in the diagnosis of narcolepsy and other hypersomnias Arch Neurol 59:1553-1562.
8. Mignot, E. 1998. Genetic and familial aspects of narcolepsy. Neurology 50:S16-22.
9. Hallmayer, J., Faraco, J., Lin, L., Hesselson, S., Winkelmann, J., Kawashima, M., Mayer, G., Plazzi, G., Nevsimalova, S., Bourgin, P., et al. 2009. Narcolepsy is strongly associated with the T-cell receptor alpha locus. Nat Genet 41:708-711.
10. Smith, A. J., Jackson, M. W., Neufing, P., McEvoy, R. D., and Gordon, T. P. 2004. A functional autoantibody in narcolepsy. Lancet 364:2122-2124.
11. Jackson, M. W., Reed, J. H., Smith, A. J., and Gordon, T. P. 2008. An autoantibody in narcolepsy disrupts colonic migrating motor complexes. J Neurosci 28:13303-13309.
12. Overeem, S., Black, J. L., 3rd, and Lammers, G. J. 2008. Narcolepsy: immunological aspects. Sleep Med Rev 12:95-107.
13. Crocker, A., Espana, R. A., Papadopoulou, M., Saper, C. B., Faraco, J., Sakurai, T., Honda, M., Mignot, E., and Scammell, T. E. 2005. Concomitant loss of dynorphin, NARP, and orexin in narcolepsy. Neurology 65:1184-1188.
14. Zhang, Y., Davis, J. L., and Li, W. 2005. Identification of tribbles homolog 2 as an autoantigen in autoimmune uveitis by phage display. Mol Immunol 42:1275-1281.
15. Maret, S., Dorsaz, S., Gurcel, L., Pradervand, S., Petit, B., Pfister, C., Hagenbuchle, O., O'Hara, B. F., Franken, P., and Tafti, M. 2007. Homerla is a core brain molecular correlate of sleep loss. Proceedings of the National Academy of Sciences of the United States of America 104:20090-20095.
16. Yuan, H., Zhang, P., Qin, L., Chen, L., Shi, S., Lu, Y., Yan, F., Bai, C., Nan, X., Liu, D., et al. 2008. Overexpression of SPINDLIN1 induces cellular senescence, multinucleation and apoptosis. Gene 410:67-74.
17. Backberg, M., Ultenius, C., Fritschy, J. M., and Meister, B. 2004. Cellular localization of GABA receptor alpha subunit immunoreactivity in the rat hypothalamus: relationship with neurones containing orexigenic or anorexigenic peptides. J Neuroendocrinol 16:589-604.
18. Honda, M., Eriksson, K. S., Zhang, S., Tanaka, S., Lin, L., Salehi, A., Hesla, P. E., Maehlen, J., Gaus, S. E., Yanagisawa, M., et al. 2009. IGFBP3 colocalizes with and regulates hypocretin (orexin). PLoS One 4:e4254.
19. Tang, W. M., Pulido, J. S., Eckels, D. D., Han, D. P., Mieler, W. F., and Pierce, K. 1997. The association of HLA-DR15 and intermediate uveitis. Am J Ophthalmol 123:70-75.
20. Thannickal, T. C., Nienhuis, R., and Siegel, J. M. 2009. Localized loss of hypocretin (orexin) cells in narcolepsy without cataplexy. Sleep 32:993-998.
21. Lecendreux, M., Maret, S., Bassetti, C., Mouren, M. C., and Tafti, M. 2003. Clinical efficacy of high-dose intravenous immunoglobulins near the onset of narcolepsy in a 10-year-old boy. J Sleep Res 12:347-348.
22. Dauvilliers, Y., Carlander, B., Rivier, F., Touchon, J., and Tafti, M. 2004. Successful management of cataplexy with intravenous immunoglobulins at narcolepsy onset. Ann Neurol 56:905-908.
23. Dauvilliers, Y., Abril, B., Mas, E., Michel, F., and Tafti, M. 2009. Normalization of hypocretin-1 in narcolepsy after intravenous immunoglobulin treatment. Neurology 73:1333-1334.
24. Roy, P. J., Stuart, J. M., Lund, J., and Kim, S. K. 2002. Chromosomal clustering of muscle-expressed genes in Caenorhabditis elegans. Nature 418:975-979.
25. Yang, Z., Edenberg, H. J., and Davis, R. L. 2005. Isolation of mRNA from specific tissues of Drosophila by mRNA tagging. Nucleic Acids Res 33:e148.
26. Bolstad, B. M., Irizarry, R. A., Astrand, M., and Speed, T. P. 2003. A comparison of normalization methods for high density oligonucleotide array data based on variance and bias. Bioinformatics 19:185-193.
27. Gautier, L., Cope, L., Bolstad, B. M., and Irizarry, R. A. 2004. affy—analysis of Affymetrix GeneChip data at the probe level. Bioinformatics 20:307-315.
28. Smyth, G. K. 2004. Linear models and empirical bayes methods for assessing differential expression in microarray experiments. Stat Appl Genet Mol Biol 3:Article3.
29. Dauvilliers, Y., Montplaisir, J., Molinari, N., Carlander, B., Ondze, B., Besset, A., and Billiard, M. 2001. Age at onset of narcolepsy in two large populations of patients in France and Quebec. Neurology 57:2029-2033.
30. Fischer A, von Eiff C, Kuczius T, Omoe K, Peters G, Becker K. A quantitative real-time immuno-PCR approach for detection of staphylococcal enterotoxins. J Mol Med. 2007 May; 85(5):461-9.
31. Lind K, Kubista M Development and evaluation of three real-time immuno-PCR assemblages for quantification of PSA. J Immunol Methods. 2005 September; 304(1-2):107-16.
32. Zhang H T, Kacharmina J E, Miyashiro K, Greene M I, Eberwine J. Protein quantification from complex protein mixtures using a proteomics methodology with single-cell resolution. Proc Nail Acad Sci USA. 2001 May 8; 98(10):5497-502.
33. Adler M, Wacker R, Niemeyer C M. Biochem Biophys Res Commun. 2003 Aug. 22; 308(2):240-50. A real-time immuno-PCR assay for routine ultrasensitive quantification of proteins.
34. Niemeyer C M, Adler M, Wacker R. Trends Biotechnol. 2005 April; 23(4):208-16. Immuno-PCR: high sensitivity detection of proteins by nucleic acid amplification.

1. A method of diagnosing a neurological disorder with immune reaction in a mammal comprising the step of detecting, in a test sample from a mammal, autoantibodies to an epitope of at least one Trib2 protein or an antigenic determinant thereof.

2. The method of claim 1 comprising the steps of a) contacting the test sample with the at least one Trib2 protein or an antigenic determinant thereof; and b) detecting any antibody-antigen complexes formed between the at least one Trib2 protein or an antigenic determinant thereof and antibodies present in the test sample, wherein presence of said complex is indicative of a neurological disorder with immune reaction.

3. The method of claim 2 wherein the neurological disorder with immune reaction is selected from the group consisting of narcolepsy with and without cataplexy, idiopathic hypersomnia, Kleine Levin syndrome, multiple sclerosis, Parkinson's disease, Alzheimer's disease, Guillain-Barré syndrome, uveitis, Myasthenia gravis, Neuromyotonia, Neuropathic diabetes, Schizophrenia, and other neuroinflammatory disorders.

4. The method of claim 1 further comprising the step of detecting orexin A protein and/or orexin B protein in a test sample from said mammal.

5. The method of claim 1 further comprising the step of assessing the clinical symptoms of the mammal.

6. The method of claim 1 wherein the mammal is a human.

7. The method of claim 1 wherein the test sample is selected from the group consisting of plasma, serum, whole blood, urine, sweat, lymph, faeces, tears, nasal fluid or nasal neuroepithelium, and cerebrospinal fluid.

8. The method of claim 2 wherein the antibody-antigen complexes are determined using an immunoassay or a chemiluminescent assay.

9. A method of treating and/or preventing a neurological disorder with immune reaction in patient comprising the steps of:
i) assessing the clinical symptoms corresponding to a neurological disorder with immune reaction in the patient;
ii) detecting the presence or absence of autoantibodies to an epitope of at least one Trib2 protein or an antigenic determinant thereof in a test sample obtained from the patient according to the method of claim 1; and
iii) administering to a patient in need thereof a compound inactivating the autoantibody.

10. The method of claim 9 further comprising the step of detecting orexin A protein and/or orexin B protein in a test sample from said mammal.

11. The method of treating and/or preventing a neurological disorder with immune reaction of claim 9 wherein the compound that inactivates the autoantibody is selected from the group consisting of beta interferons, interleukin-12, corticosteroids, plasmaphoresis, and intravenous immunoglobulines such as an immunoglobuline G, or a combination of said compounds, or pharmaceutical compositions.

12. An assay kit for diagnosing a neurological disorder with immune reaction in a mammal comprising i) at least one epitope of at least one Trib2 protein or an antigenic determinant thereof; ii) reagents for the constitution of the medium appropriate for carrying out the autoantibody-epitope reaction; and iii) the reagents making possible the detection of the complex formed.

13. A method of monitoring treatment in a patient, suffering from a neurological disorder with immune reaction and treated by administration of a compound, or a pharmaceutical composition, the method comprising the steps of:
(a) determining the concentration or amount in a first test sample from a patient of one or more autoantibodies reactive with Trib2 protein, or an antigenic determinant thereof;
(b) treating the patient with one or more pharmaceutical compositions for a period of time;
(c) determining the concentration or amount in a second or subsequent test sample obtained from the patient following treatment in step (b) of one or more autoantibodies reactive with Trib2 protein, or an antigenic determinant thereof; and
(d) comparing the concentration or amount of one or more autoantibodies reactive with Trib2 protein or an antigenic determinant thereof determined in step (c) with the concentration or amount of one or more autoantibodies reactive with Trib2 protein or an antigenic determinant thereof determined in step (a), wherein if the concentration or amount determined in step (c) is unchanged or is unfavorable when compared to the concentration or amount of one or more autoantibodies reactive with Trib2 protein or an antigenic determinant thereof determined in step (a), then the disorder in the subject is determined to have continued, progressed or worsened, and the subject should be treated with a higher concentration of the one or more pharmaceutical compositions administered to the subject in step (b) or the subject should be treated with one or more pharmaceutical compositions that are different then the one or more pharmaceutical compositions administered to the subject in step (b), further wherein, if the concentration or amount of one or more autoantibodies reactive with Trib2 protein or an antigenic determinant thereof determined in step (c) is favorable when compared to the concentration or amount of one or more autoantibodies reactive with Trib2 protein or an antigenic determinant thereof determined in step (a), then the disorder in the subject is determined to have discontinued, regressed or improved, and the subject should continue to be administered the one or pharmaceutical compositions of step (b).

14. A method of monitoring treatment in a patient, suffering from a neurological disorder with immune reaction and treated by administration of a compound, or a pharmaceutical composition, the method comprising the steps of:
(a) determining the concentration or amount of orexin A protein and/or orexin B protein in said first, or another, test sample from a patient;
(b) treating the patient with one or more pharmaceutical compositions for a period of time;
(c) determining the concentration or amount of orexin A protein and/or orexin B protein in said second, or another, test sample from a patient; and
(d) comparing the concentration or amount of orexin A protein and/or orexin B protein determined in step (c) with the concentration or amount of orexin A protein and/or orexin B protein determined in step (a), wherein if the concentration or amount determined in step (c) is unchanged or is unfavorable when compared to the concentration or amount determined in step (a), then the disorder in the subject is determined to have continued, progressed or worsened, and the subject should be treated with a higher concentration of the one or more pharmaceutical compositions administered to the subject in step (b) or the subject should be treated with one or more pharmaceutical compositions that are different then the one or more pharmaceutical compositions administered to the subject in step (b), further wherein, if the concentration or amount of orexin A protein and/or orexin B protein determined in step (c) is favorable when compared to the concentration or amount determined in step (a), then the disorder in the subject is determined to have discontinued, regressed or improved, and the subject should continue to be administered the one or pharmaceutical compositions of step (b).


SEQUENCE LISTING (15 sequences)

SEQ ID NO: 1 - protein, 343 aa, Homo sapiens
MNIHRSTPITIARYGRSRNKTQDFEELSSIRSAEPSQSFSPNLGSPSPPETPNLSHCVSCIGKYLLLEPLEGDHVFRAVHLHSGEELVCKVFDISCYQESLAPCFCLSAHSNINQITEIILGETKAYVFFERSYGDMHSFVRTCKKLREEEAARLFYQIASAVAHCHDGGLVLRDLKLRKFIFKDEERTRVKLESLEDAYILRGDDDSLSDKHGCPAYVSPEILNTSGSYSGKAADVWSLGVMLYTMLVGRYPFHDIEPSSLFSKIRRGQFNIPETLSPKAKCLIRSILRREPSERLTSQEILDHPWFSTDFSVSNSAYGAKEVSDQLVPDVNMEENLDPFFN

SEQ ID NO: 2 - protein, 28 aa, Homo sapiens
NSAYGAKEVSDQLVPDVNMEENLDPFFN

SEQ ID NO: 3 - DNA, 22 bp, Artificial Sequence
  Synthesized sequence
aagccatgcacccttctcttgc

SEQ ID NO: 4 - DNA, 21 bp, Artificial Sequence
  Synthesized sequence
ccttattccaagcggcttcgg

SEQ ID NO: 5 - DNA, 22 bp, Artificial Sequence
  Synthesized sequence
gagctggacggcgacgtaaacg

SEQ ID NO: 6 - DNA, 23 bp, Artificial Sequence
  Synthesized sequence
aggaccatgtgatcgcgcttctc

SEQ ID NO: 7 - DNA, 25 bp, Artificial Sequence
  Synthesized sequence
agagattttgacatgctcaggtgta

SEQ ID NO: 8 - DNA, 20 bp, Artificial Sequence
  Synthesized sequence
gcggaccagcaggtatcaga

SEQ ID NO: 9 - DNA, 28 bp, Artificial Sequence
  Synthesized sequence
ctgggaagagtctaccgaccatgttggc

SEQ ID NO: 10 - DNA, 27 bp, Artificial Sequence
  Synthesized sequence
taaagatcatgacatcgattacaagga

SEQ ID NO: 11 - DNA, 20 bp, Artificial Sequence
  Synthesized sequence
aggtcccccacgtacagaga

SEQ ID NO: 12 - DNA, 19 bp, Artificial Sequence
  Synthesized sequence
acgatgacaagctcgacat

SEQ ID NO: 13 - DNA, 18 bp, Artificial Sequence
  Synthesized sequence
ctgctgcccgacaaccac

SEQ ID NO: 14 - DNA, 20 bp, Artificial Sequence
  Synthesized sequence
accatgtgatcgcgcttctc

SEQ ID NO: 15 - DNA, 23 bp, Artificial Sequence
  Synthesized sequence
ccagtccgccctgagcaaagacc